(19) United States
(12) Patent Application Publication
(10) Pub. No.: US 2007/0179318 A1
(43) Pub. Date: Aug. 2, 2007

(54) Aromatic amine derivative and organic electroluminescence device using same

(75) Inventors: Masahiro Kawamura, Chiba (JP); Nobuhiro Yabunouchi, Chiba (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Idemitsu Kosan Co., Ltd., Chiyoda-ku (JP)

(21) Appl. No.: 11/411,141

(22) Filed: Apr. 26, 2006

(30) Foreign Application Priority Data
Jun. 15, 2005 (JP) 2005-175473

(51) Int. Cl. C07C 211/54; C07C 211/57; C09K 11/06; H01L 51/54

(52) U.S. Cl. 564/434; 428/690; 428/917; 313/504; 313/506; 257/E51.051; 564/433; 564/427

(57) ABSTRACT

An aromatic amine compound having a specific structure, the following organic electroluminescence device and the noble aromatic amine compounds enable for realizing the device are provided. The device, which comprises at least one organic thin film layer comprising a light emitting layer sandwiched between a pair of electrode consisting of an anode and a cathode, wherein at least one of the organic thin film layers comprises the aromatic amine derivative singly or as its mixture component, exhibits various luminescent hue and has high heat resistance, a long lifetime, high luminance and high current efficiency. In particular, the device exhibits low decay of luminance based on driving it.

### TECHNICAL FIELD

The present invention relates to an aromatic amine derivative and an organic electroluminescence device including the aromatic amine derivative, in particular, to an organic electroluminescence device exhibiting various luminescent hue, and having high heat resistance, a long lifetime, high luminance and high current efficiency, and also to the novel aromatic amine derivative for realizing the organic electroluminescence device.

### BACKGROUND ART

An organic electroluminescence (“electroluminescence” will be occasionally referred to as “EL”, hereinafter) device is a spontaneous light emitting device which utilizes the principle that a fluorescent substance emits light by energy of recombination of holes injected from an anode and electrons injected from a cathode when an electric field is applied.

Since an organic EL device of the laminate type driven under a low electric voltage was reported by C. W. Tang et al. of Eastman Kodak Company (C. W. Tang and S. A. Vanslyke, Applied Physics Letters, Volume 51, Pages 913, 1987), many studies have been conducted on organic EL devices using organic materials as the constituting materials. Tang et al. used a laminate structure using tris(8-quinolinolato) aluminum for the light emitting layer and a triphenyldiamine derivative for the hole transporting layer.

Advantages of the laminate structure are that the efficiency of hole injection into the light emitting layer can be increased, that the efficiency of forming excitons which are formed by blocking and recombining electrons injected from the cathode can be increased, and that excitons formed among the light emitting layer can be enclosed.

As the structure of the organic EL device, a two-layered structure having a hole transporting (injecting) layer and an electron transporting and light emitting layer and a three-layered structure having a hole transporting (injecting) layer, a light emitting layer and an electron transporting (injecting) layer are well known. To increase the efficiency of recombination of injected holes and electrons in the devices of the laminate type, the structure of the device and the process for forming the device have been studied.

So far, the aromatic diamine derivatives described in Patent literature 1 and the aromatic condensed ring diamine derivatives described in Patent literature 2 have been known as a hole transporting material.

When an organic EL device is driven or stored at high temperature, there causes many adverse affects such as sifting emitted color, lowing current efficiency, rising driving voltage, shortening the lifetime and the like. In order to avoid the above, it has been required that the glass transition temperature (Tg) of a hole transportation material should be high.

For example, the tetramers of the aromatic amines disclosed in Patent literatures 3, 4 and 5 have been known as a hole transporting material with high Tg. However, these materials are very hardly soluble and purification thereof is difficult. Therefore, the organic EL devices employed these materials had significant decay of luminance of the organic EL device based on driving them, and in particular, the device emitting blue light was notable in decaying luminance based on driving the organic EL devices.
[Patent literature 1] U.S. Pat. 4,720,432
[Patent literature 2] U.S. Pat. 5,061,569
[Patent literature 3] U.S. Pat. 3,220,950
[Patent literature 4] U.S. Pat. 3,194,657
[Patent literature 5] U.S. Pat. 3,180,802

### DISCLOSURE OF THE INVENTION

The present invention has been made so as to solve the aforementioned problems, and its objective is to provide an organic EL device exhibiting various luminescent hue, and having high heat resistance, a long lifetime, high luminance and high current efficiency, in particular, an organic EL device enables to avoid decay of luminance of the organic EL device based on driving it, and also to the novel aromatic amine derivative for realizing the organic EL device.

As a result of intensive researches and studies to achieve the above objective by the present inventors, it was found that employing an aromatic amine derivative represented by the following general formula (I) enables to avoid decay of luminance based on driving an organic EL device. In other word, the present invention provides an aromatic amine derivative represented by the following general formula (I):

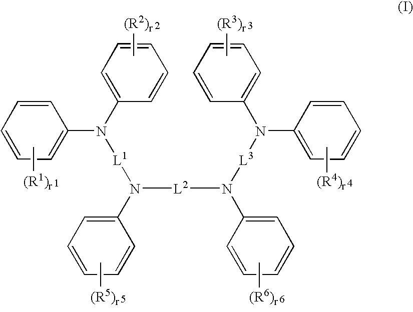

In addition, the above objective has been achieved by an organic EL device comprising at least one of organic thin film layers including a light emitting layer interposed between a pair of electrodes consisting of an anode and a cathode, wherein at least one of the organic thin film layers contains one selected from the aforementioned aromatic amine derivatives represented by the general formula (I) singly or as a component of mixture thereof.

An organic EL device including the aromatic amine derivatives of the present invention exhibits various luminescent hue and has high heat resistance. In particular, employing an aromatic amine derivative of the present invention as a hole injecting/transportating material of an organic EL device enables to provide the organic EL device having a long lifetime, high luminance and high current efficiency, and also to avoid decay of luminance based on driving the organic EL device.

The first invention is an aromatic amine derivative represented by the following general formula (I).

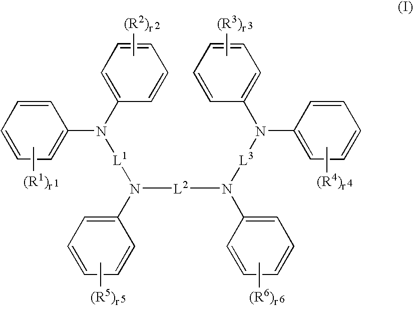

In the general formula (I), R¹ to R⁶ each independently represents a substituted or unsubstituted alkyl group having carbon atoms of 1 to 6, or a substituted or unsubstituted aryl group having nuclear carbon atoms of 6 to 20. In the general formula (I), L¹ to L³ each independently represents an linking group represented by the general formula (II).

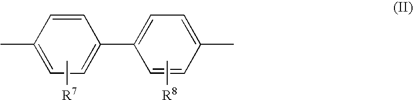

In the general formula (II), R⁷ and R⁸ each independently represents a hydrogen atom, a substituted or unsubstituted alkyl group having carbon atoms of 1 to 6, or a substituted or unsubstituted aryl group having nuclear carbon atoms of 6 to 20. In addition, R⁷ and R⁸ may bond each other to form a saturated or unsaturated ring.

Examples of a substituted or unsubstituted alkyl group having carbon atoms of 1 to 6 of R¹ to R⁸ in the general formulae (I) and (II) include methyl group, ethyl group, n-propyl group, isopropyl group, n-butyl group, s-butyl group, t-butyl group, n-pentyl group, cyclopentyl group, n-hexyl group and cyclohexyl group.

Examples of a substituted or unsubstituted aryl group having nuclear carbon atoms of 6 to 20 of R¹ to R⁸ in the general formulae (I) and (II) include phenyl group, 1-naphthyl group, 2-naphthyl group, 1-anthryl group, 2-anthryl group, 9-anthryl group, 1-phenanthryl group, 2-phenanthryl group, 3-phenanthryl group, 4-phenanthryl group, 9-phenanthryl group, 1-naphthacenyl group, 2-naphthacenyl group, 9-naphthacenyl group, 1-pyrenyl group, 2-pyrenyl group, 4-pyrenyl group, 2-biphenylyl group, 3-biphenylyl group, 4-biphenylyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-tolyl group, m-tolyl group, p-tolyl group, p-t-butylphenyl group, p-(2-phenylpropyl) phenyl group, 3-methyl-2-naphthyl group, 4-methyl-1-naphthyl group, 4-methyl-1-anthryl group, 4′-methylbiphenylyl group, 4″-t- butyl-p-terphenyl-4-yl group, fluorenyl group and the like. Phenyl group, naphthyl group, biphenyl group, anthryl group, phenanthryl group, pyrenyl group, chrysenyl group and fluorenyl group are preferable. Phenyl group and naphthyl group are particularly preferable.

L¹ to L³ in the general formula (I) each independently may be selected from linking groups consisting of the following general formulae (II-1) to (II-4):

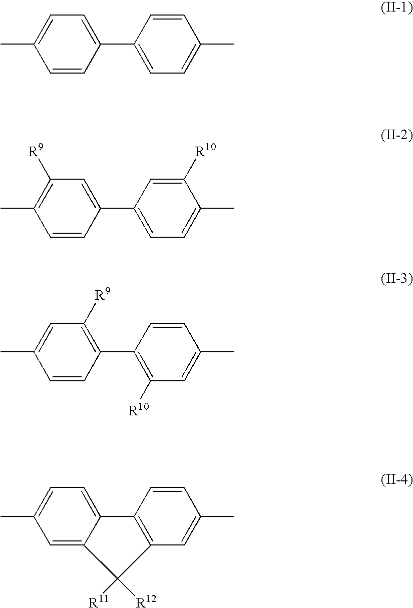

R⁹ to R¹² in the general formulae (II-1) to (II-4) each independently represents a substituted or unsubstituted alkyl group having carbon atoms of 1 to 6, or a substituted or unsubstituted aryl group having nuclear carbon atoms of 6 to 20. Examples thereof are the same with ones of R⁷ and R⁸. In addition, R⁹ and R¹⁰, and R¹¹ and R¹² respectively may bond each other to form a saturated or unsaturated ring.

r¹ to r⁶ each independently represents an integer of 0 to 5, and r¹+r²+r³+r⁴+r⁵+r⁶≧1. Further, when any one of r¹ to r⁶ is 2 or larger, each of R¹ to R⁶ corresponding thereto may be the same with or different from the other. However, at least one of R¹ to R⁶ represents a substituted or unsubstituted aryl group having nuclear carbon atoms of 6 to 20.

An alkyl group and/or an aryl group in the general formulae of (I), (II) and (II-1) to (II-4) may have substituent, and examples thereof include an alkyl group having carbon atoms of 1 to 10 such as methyl group, ethyl group, isopropyl group, n-propyl group, n-butyl group, s-butyl group, t-butyl group, n-pentyl group, cyclopentyl group, n-hexyl group and cyclohexyl group, an alkoxy group having carbon atoms of 1 to 10 such as methoxy group, ethoxy group, isopropoxy group, n-propoxy group, s-butoxy group, t-butoxy group, pentoxy group, hexyloxy group, cyclopentoxy group and cyclohexyloxy group. Among those, an alkyl group having carbon atoms of 1 to 10 are preferable, and methyl group, ethyl group, isopropyl group, n-propyl group, n-butyl group, s-butyl group, t-butyl group, n-pentyl group, cyclopentyl group, n-hexyl group and cyclohexyl group are particularly preferable. Most preferable examples of the aromatic amine compounds having substituent include the following.

The present invention provides a method of employing the aromatic amine derivatives represented by the general formula (I) as a material for an organic electroluminescence device.

Further, the present invention provide an organic electroluminescence device comprising at least one of organic thin film layers including a light emitting layer interposed between a pair of electrode consisting of an anode and a cathode, wherein at least one of the organic thin film layers contains one selected from the aforementioned aromatic amine derivatives represented by the general formula (I) singly or as a component of mixture thereof, therefore the above objective has been achieved.

The present invention provides an organic electroluminescence device comprising a hole transporting zone containing the aforementioned aromatic amine derivatives, an organic electroluminescence device comprising a hole transporting layer containing the aforementioned aromatic amine derivatives, an organic electroluminescence device comprising a hole transporting layer containing primarily the aforementioned aromatic amine derivatives represented by the general formula (I), an organic electroluminescence device comprising layers of a hole transporting layer containing the aforementioned aromatic amine derivatives represented by the general formula (I) and a light emitting layer comprising a phosphorescent metal complex and a host material, and an organic electroluminescence device emitting blue light.

Examples of the general formula (I) are shown as follows, but not limited thereto. Here, Me in the examples represents a methyl group.

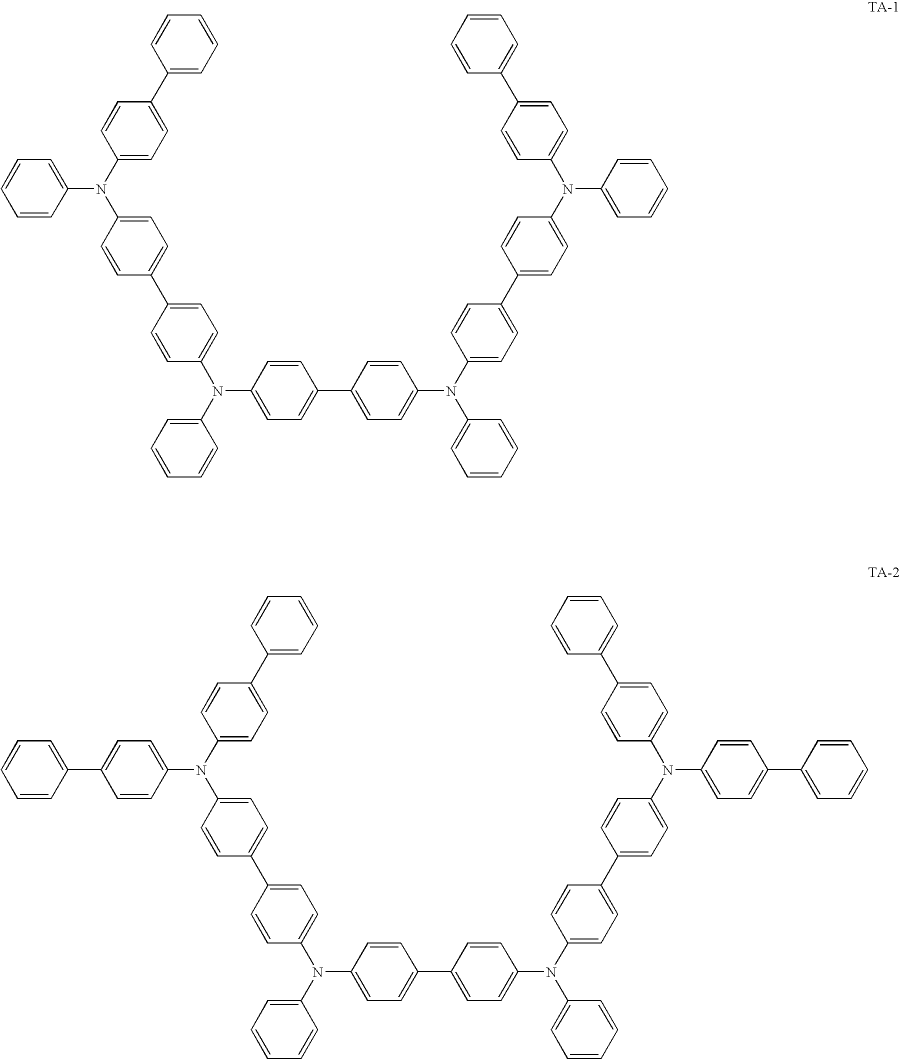

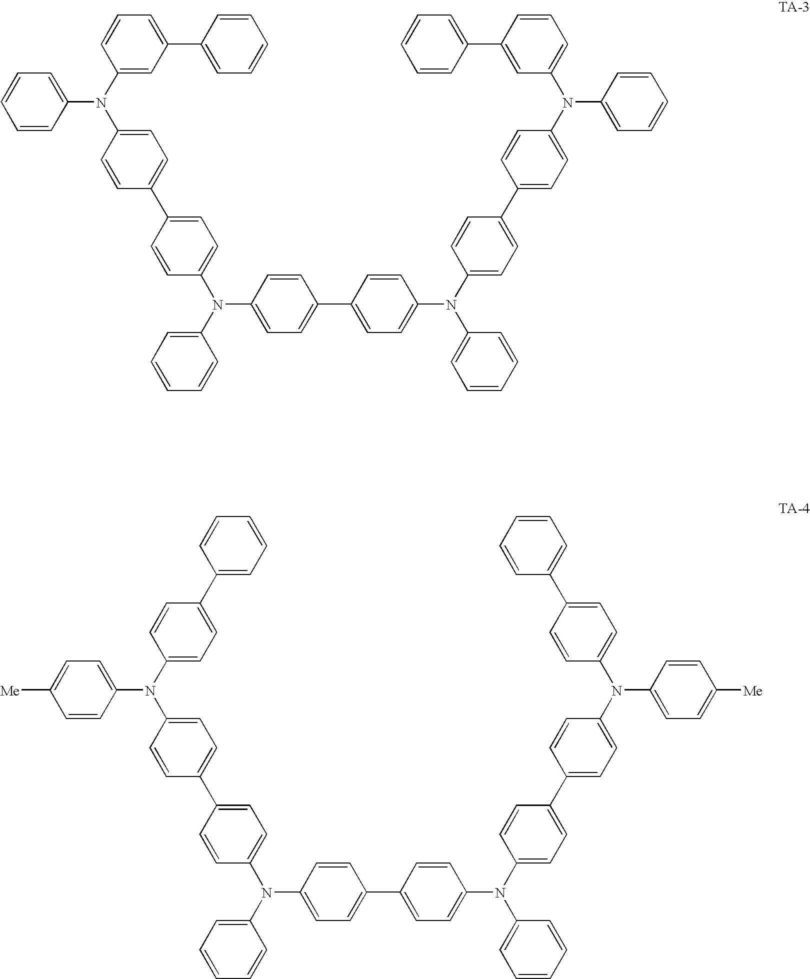

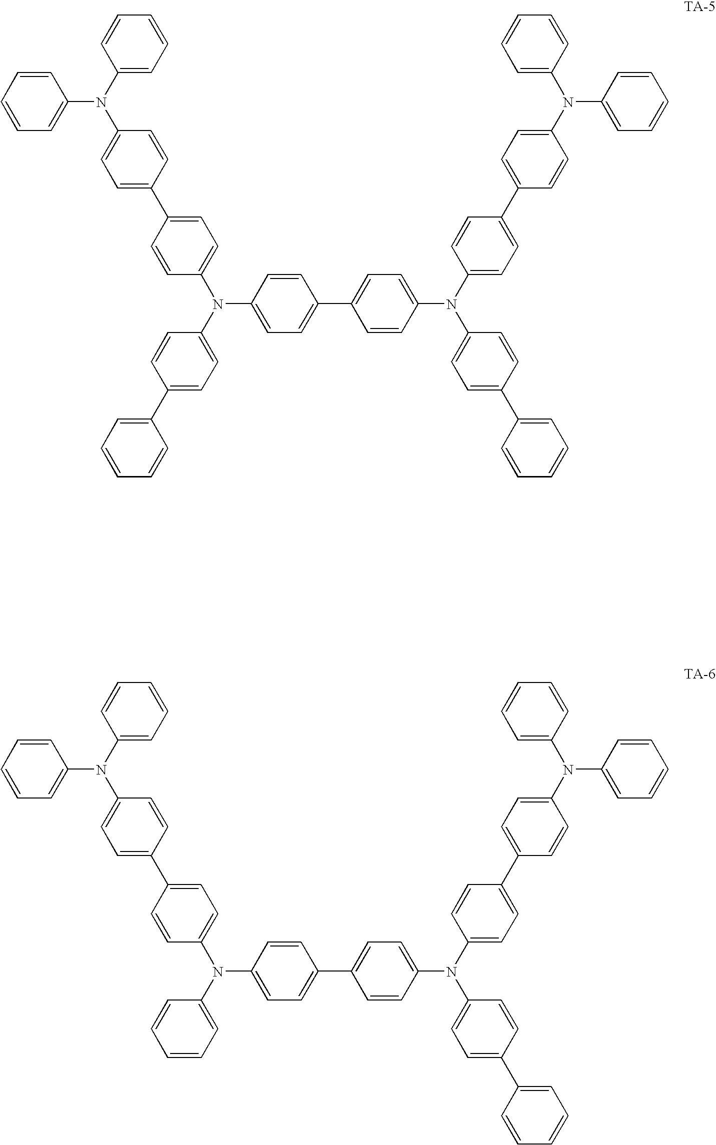

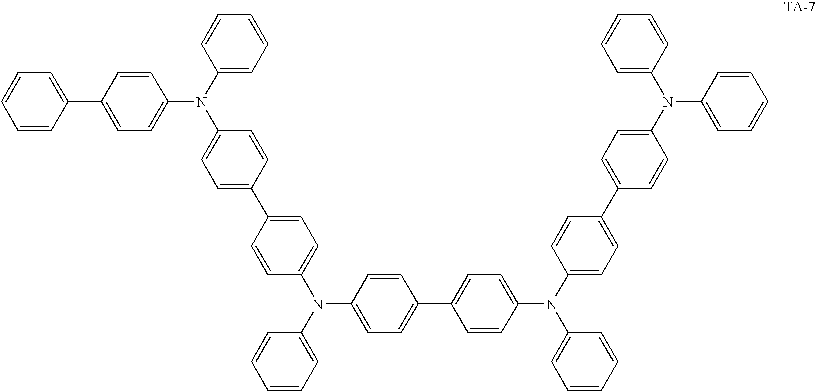

The second invention enables an organic EL device to contain the aromatic amine derivatives as singly or as a mixture thereof The aromatic amine derivatives are employed preferably for a hole transporting zone, and it is possible to obtain an excellent organic EL device when they are employed more preferably for a hole transporting layer.

The following is a detail description of the organic EL device of the present invention.

I. Construction of Organic EL Device

The following is typical examples of the construction of the organic EL device of the present invention, but not limited thereto.
(1) an anode/a light emitting layer/a cathode;
(2) an anode/a hole injecting layer/a light emitting layer/a cathode;
(3) an anode/a light emitting layer/an electron injecting layer/a cathode;
(4) an anode/a hole injecting layer/a light emitting layer/an electron injecting layer/a cathode;
(5) an anode/an organic semiconductor layer/a light emitting layer/a cathode;
(6) an anode/an organic semiconductor layer/an electron barrier layer/a light emitting layer/a cathode;
(7) an anode/an organic semiconductor layer/a light emitting layer/an adhesion improving layer/a cathode;
(8) an anode/a hole injecting layer/a hole transporting layer/a light emitting layer/an electron injecting layer/a cathode;
(9) an anode/an insulating layer/a light emitting layer/an insulating layer/a cathode;
(10) an anode/an inorganic semiconductor layer/an insulating layer/a light emitting layer/an insulating layer/a cathode;
(11) an anode/an organic semiconductor layer/an insulating layer/a light emitting layer/an insulating layer/a cathode;
(12) an anode/an insulating layer/a hole injecting layer/a hole transporting layer/a light emitting layer/an insulating layer/a cathode; and
(13) an anode/an insulating layer/a hole injecting layer/a hole transporting layer/a light emitting layer/an electron injecting layer/a cathode.

Among those, the construction (8) is generally employed in particular. Although the compounds of the present invention may be used for any one of the above organic layers, it is preferable for. them to be used for a light emitting zone or a hole transportation zone among those construction elements. It is preferable that the compounds are contained in a hole transporting layer. Amount to be contained therein may be selected from the range of 30 to 100 mole %.

II. Transparent Substrate

The organic EL device of the present invention is produced on a transparent substrate. It is preferable that the substrate which transmits light has a transmittance of light of 50% or greater in the visible region of 400 to 700 nm. It is also preferable that a flat and smooth substrate is employed.

As the transparent substrate, for example, glass sheet and synthetic resin sheet are advantageously employed. Specific examples of the glass sheet include soda-lime glass, glass containing barium and strontium, lead glass, aluminosilicate glass, borosilicate glass, barium borosilicate glass, quartz and the like. In addition, specific examples of the synthetic resin sheet include sheet made of polycarbonate resins, acrylic resins, polyethylene terephthalate resins, polyether sulfide resins, polysulfone resins and the like.

III. Anode

The anode in the organic EL device of the present invention has the function of injecting holes into a hole transport layer or into a light emitting layer, and it is effective that the anode has a work function of 4.5 eV or greater. Specific examples of the material for the anode used in the present invention include indium tin oxide alloy (ITO), tin oxide (NESA), gold, silver, platinum, copper, lanthanoid and the like. In addition, alloys or laminates thereof may be used.

The anode can be prepared by forming a thin film of the electrode material described above in accordance with a process such as a vapor deposition process or a sputtering process.

When the light emitted from the light emitting layer is obtained through the anode, it is preferable that the anode has a transmittance of the emitted light greater than 10%. It is also preferable that the sheet resistivity of the anode is several hundred Ω/□ or smaller. The thickness of the anode is, in general, selected in the range of from 10 nm to 1 μm and preferably in the range of from 10 to 200 nm, although the preferable range may be different depending on the used material.

IV Light Emitting Layer

In the organic EL device of the present invention, the light emitting layer has the following functions. Namely,
(1) The injecting function: the function of injecting holes from the anode or the hole injecting layer and injecting electrons from the cathode or the electron injecting layer when an electric field is applied;
(2) The transporting function: the function of transporting injected charges (electrons and holes) by the force of the electric field; and
(3) The light emitting function: the function of providing the field for recombination of electrons and holes and leading the recombination to the emission of light.

There may be a difference between the capability of the holes being injected and the capability of the electrons being injected. The ability of transportation expressed by the mobility may be different between holes and electrons. It is preferable that either one of the charges is transferred.

As the process for forming the light emitting layer, a well known process such as the vapor deposition process, the spin coating process and the LB process can be employed. It is preferable that a light emitting layer is a molecular sedimentation film particularly.

Here, the molecular sedimentation film is defined as a thin film formed by sedimentation of a material compound in the gas phase or a thin film formed by solidification of a material compound in a solution or in the liquid phase. The molecular sedimentation film may be differentiated from a thin film (a molecular build-up film) formed by the LB process, base on the differences between aggregation structures and higher-order structures, and also the differences resulting from functionalities thereof.

In addition, as disclosed in Japanese Patent Laid-open No. Showa 57(1982)-51781, a light emitting layer can also be formed by dissolving a material compound with a binder such as resin in a solvent and preparing a thin film in accordance with the spin coating and the like from the solution.

In the present invention, any well known light emitting material other than a light emitting material consisting of an asymmetric aromatic amine derivative of the present invention may be optionally contained in the light emitting layer; or a light emitting layer containing other well known light emitting material may be laminated with the light emitting layer containing the light emitting material of the present invention each in an extent of not obstructing to achieve the objective of the present invention respectively.

A known light emitting material having a condensed aromatic ring such as anthracene and pyrene is particularly preferable. Specific examples thereof include an anthracene derivative, an asymmetric monoanthracene derivative, an asymmetric anthracene derivative, an asymmetric pyrene derivative and the like as follows.

The anthracene derivatives as known light emitting materials include the following;

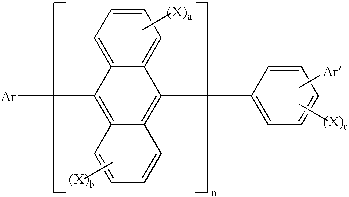

In the above general formula, Ar represents a substituted or unsubstituted condensed aromatic group having nuclear carbon atoms of 10 to 50. Ar′ represents a substituted or unsubstituted aromatic group having nuclear carbon atoms of 6 to 50. X represents a substituted or unsubstituted aromatic group having nuclear carbon atoms of 6 to 50, a substituted or unsubstituted aromatic heterocyclic group having nuclear atoms of 5 to 50, a substituted or unsubstituted alkyl group having carbon atoms of 1 to 50, a substituted or unsubstituted alkoxy group having carbon atoms of 1 to 50, a substituted or unsubstituted aralkyl group having carbon atoms of 6 to 50, a substituted or unsubstituted aryloxy group having nuclear carbon atoms of 5 to 50, a substituted or unsubstituted arylthio group having nuclear carbon atoms of 5 to 50, a substituted or unsubstituted alkoxycarbonyl group having carbon atoms of 1 to 50, a caboxyl group, a halogen atom, a cyano group, a nitro group or a hydroxyl group. a, b and c each represents an integer of 0 to 4.n represents an integer of 1 to 3. In addition, when n represents 2 or larger, the anthracene core within [ ] may be the same with or different from the other.

The asymmetric monoanthracene derivatives as known light emitting materials include the following;

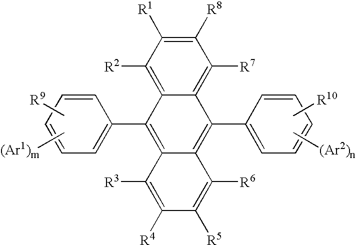

In the above general formula, Ar¹ and Ar² each independently represents a substituted or unsubstituted aromatic ring group having nuclear carbon atoms of 6 to 50, and m and n each represents an integer of 1 to 4. However, when m=n=1 and each bonding position of Ar¹ and Ar² to each benzene ring thereof is bilaterally symmetrical each other, Ar¹ is different from Ar². When m or n represents an integer of 2 to 4, m is different from n. R¹ to R¹⁰ each independently represents a hydrogen atom, a substituted or unsubstituted aromatic ring group having nuclear carbon atoms of 6 to 50, a substituted or unsubstituted aromatic heterocyclic group having nuclear atoms of 5 to 50, a substituted or unsubstituted alkyl group having carbon atoms of 1 to 50, a substituted or unsubstituted cycloalkyl group, a substituted or unsubstituted alkoxy group having carbon atoms of 1 to 50, a substituted or unsubstituted aralkyl group having carbon atoms of 6 to 50, a substituted or unsubstituted aryloxy group having nuclear carbon atoms of 5 to 50, a substituted or unsubstituted arylthio group having nuclear carbon atoms of 5 to 50, a substituted or unsubstituted alkoxycarbonyl group having carbon atoms of 1 to 50, a substituted or unsubstituted silyl group, a caboxyl group, a halogen atom, a cyano group, a nitro group or a hydroxyl group.

The asymmetric anthracene derivatives as known light emitting materials include the following;

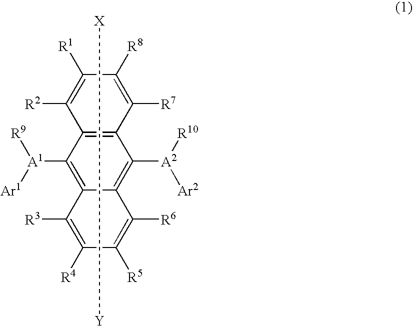

In the above general formula, A¹ and A² each independently represents a substituted or unsubstituted condensed aromatic ring group having nuclear carbon atoms of 10 to 20. Ar¹ and Ar² each independently represents a hydrogen atom or a substituted or unsubstituted aromatic ring group having nuclear carbon atoms of 6 to 50. R¹ to R¹⁰ each independently represents a hydrogen atom, a substituted or unsubstituted aromatic ring group having nuclear carbon atoms of 6 to 50, a substituted or unsubstituted aromatic heterocyclic group having nuclear atoms of 5 to 50, a substituted or unsubstituted alkyl group having carbon atoms of 1 to 50, a substituted or unsubstituted cycloalkyl group, a substituted or unsubstituted alkoxy group having carbon atoms of 1 to 50, a substituted or unsubstituted aralkyl group having carbon atoms of 6 to 50, a substituted or unsubstituted aryloxy group having nuclear atoms of 5 to 50, a substituted or unsubstituted arylthio group having nuclear atoms of 5 to 50, a substituted or unsubstituted alkoxycarbonyl group having carbon atoms of 1 to 50, a substituted or unsubstituted silyl group, a caboxyl group, a halogen atom, a cyano group, a nitro group or a hydroxyl group. Ar¹, Ar², R⁹ and R¹⁰ each may be a plural number and two neighboring groups thereof may form a saturated or unsaturated ring structure. However, it is excluded a case where the groups bonding at 9- and 10-positions of anthracene at the core in the general formula (1) are symmetrical with respect to the X-Y axis.

The asymmetric pyrene derivatives as known light emitting materials include the following;

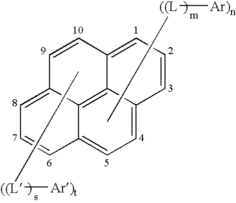

In the above general formula, Ar and Ar′each independently represents a substituted or unsubstituted aromatic group having nuclear carbon atoms of 6 to 50. L and L′ each represents a substituted or unsubstituted phenylene group, a substituted or unsubstituted naphtharenylene group, a substituted or unsubstituted fuluorenylene group or a substituted or unsubstituted dibenzosilolylene group. m represents an integer of 0 to 2, n represents an integer of 1 to 4, s represents an integer of 0 to 2 and t represents an integer of 0 to 4. Further, L or Ar bonds to any one of 1- to 5-positions of pyrene, and L′ or Ar′ bonds to any one of 6- to 10-positions thereof. However, when n+t is an even number, Ar, Ar′, L and L′ satisfy the following requirement (1) or (2):
(1) Ar≠Ar′ and/or L≠L′, wherein ≠ means that each group has a different structure from the other,
(2) when Ar=Ar′ and L=L′,
(2-1) m=s and/or n≠t, or
(2-2) when m=s and n=t,
(2-2-1) both L and L′ or pyrene bond respectively to a different position of Ar and Ar′, or
(2-2-2) when both L and L′ or pyrene bond respectively to the same position of Ar and Ar′, it is excluded a case where both L and L′ or both Ar and Ar′ bond respectively to 1 and 6, or 2 and 7 positions thereof.
   V Hole injecting/transporting Layer

The hole injecting/transporting layer is a layer which assists injection of holes into the light emitting layer and transport the holes to the light emitting zone. The layer exhibits a great mobility of holes and, in general, have an ionization energy as small as 5.5 eV or smaller. For the hole injecting/transporting layer, a material which transports holes to the light emitting layer at a small strength of the electric field is preferable. A material which exhibits, for example, a mobility of holes of at least 10⁻⁴ cm²/V sec under application of an electric field of from 104 to 10⁶ V/cm is preferable.

When the aromatic amine derivatives of the present invention are used in the hole transporting zone, they may be used singly or as a mixture with the other materials to form the hole injecting/transporting layer.

A material to be mixed with the aromatic amines of the present invention to form the hole injecting/transporting layer is not particularly limited if it has the aforementioned preferable properties, and it may be selected, as appropriate, from conventional materials for a charge transporting material of hole and known materials used for a hole injecting layer of an EL device.

Further examples include triazole derivatives (refer to U.S. Pat. No. 3,112,197, etc.), oxadiazole derivatives (refer to U.S. Pat. No. 3,189,447, etc.), imidazole derivatives (refer to Japanese Examined Patent KOKOKU No. Shou 37-16096, etc.), poly arylalkane derivatives (refer to U.S. Pat. Nos. 3,615,402, 3,820,989 and 3,542,544, Japanese Examined Patent KOKOKU Nos. Shou 45-555 and Shou 51-10983, Japanese Unexamined Patent Application Laid-Open Nos. Shou 51-93224, Shou 55-17105, Shou 56-4148, Shou 55-108667, Shou 55-156953, Shou 56-36656, etc.), pyrazoline derivatives and pyrazolone derivatives (refer to U.S. Pat. Nos. 3,180,729 and 4,278,746, Japanese Unexamined Application Patent Laid-Open Nos. Shou 55-88064, Shou 55-88065, Shou 49-105537, Shou 55-51086, Shou 56-80051, Shou 56-88141, Shou 57-45545, Shou 54-112637, Shou 55-74546, etc.), phenylenediamine derivatives (refer to U.S. Pat. No. 3,615,404, Japanese Examined Patent KOKOKU Nos. Shou 51-10105, Shou 46-3712 and Shou 47-25336, Japanese Unexamined Patent Application Laid-Open Nos. Shou 54-53435, Shou 54-110536, Shou 54-119925, etc.), arylamine derivatives (refer to U.S. Pat. Nos. 3,567,450, 3,180,703, 3,240,597, 3,658,520, 4,232,103, 4,175,961 and 4,012,376, Japanese Examined Patent KOKOKU Nos. Shou 49-35702 and Shou 39-27577, Japanese Unexamined Patent Application Laid-Open Nos. Shou 55-144250, Shou 56-119132 and Shou 56-22437, West German Patent No. 1,110,518, etc.), chalcone derivatives which is substituted with amino group (refer to U.S. Pat. No. 3,526,501, etc.), oxazole derivatives (disclosed in U.S. Pat. No. 3,257,203, etc.), styryl anthracene derivatives (refer to Japanese Unexamine Patent Application Laid-Open No. Shou 56-46234, etc.), fluorenone derivatives (refer to Japanese Unexamined Patent Application Laid-Open No. Shou 54-110837, etc.), hydrazone derivatives (refer to U.S. Pat. Nos. 3,717,462, Japanese Unexamined Patent Application Laid-Open Nos. Shou 54-59143, Shou 55-52063, Shou 55-52064, Shou 55-46760, Shou 55-85495, Shou 57-11350, Shou 57-148749, Hei 2-311591, etc.), stilbene derivatives (refer to Japanese Unexamined Patent Application Laid-Open Nos. Shou 61-210363, Shou 61-228451, Shou 61-14642, Shou 61-72255, Shou 62-47646, Shou 62-36674, Shou 62-10652, Shou 62-30255, Shou 60-93455, Shou 60-94462, Shou 60-174749, Shou 60-175052, etc.), silazane derivatives (U.S. Pat. No. 4,950,950), polysilane-based copolymers (Japanese Unexamined Patent Application Laid-Open No. Hei 2-204996), aniline-based copolymers (Japanese Unexamined Patent Application Laid-Open No. Hei 2-282263), an electroconductive polymer oligomer (particularly, thiophene oligomer) which is disclosed in Japanese Unexamined Patent Application Laid-Open No Hei 1-211399, etc.

With regard to the material of the hole injecting layer, the above materials are also employable, however, porphyrin compounds (published in Japanese Unexamined Patent Application Laid-Open Nos. Shou 63-2956965, etc.), aromatic tertiary amine compounds and styryl amine compounds (refer to U.S. Pat. No. 4,127,412, Japanese Unexamined Patent Application Laid-Open Nos. Shou 53-27033, Shou 54-58445, Shou 54-149634, Shou 54-64299, Shou 55-79450, Shou 55-144250, Shou 56-119132, Shou 61-295558, Shou 61-98353, Shou 63-295695, etc.) are preferable and the aromatic tertiary amine compounds are particularly preferable.

Further examples include, for example, 4,4′-bis(N-(1-naphthyl)-N-phenylamino)biphenyl (abbreviated as NPD hereunder) having two fused aromatic rings in its molecule described in U.S. Pat. No. 5,061,569, 4,4′,4″-tris(N-(3-methylphenyl)-N-phenylamino)triphenyl amine (abbreviated as MTDATA hereunder) made by connecting three triphenyl amine units to form a star burst type and the like.

Further, in addition to the aforementioned aromatic dimethylidine-based compound described as a material for the light emitting layer, inorganic compound such as p-type silicon, p-type silicon carbide or the like is employable as the material for the hole injecting layer.

To form the hole injecting/transporting layer, a thin film may be formed from the aforementioned materials for the hole injecting/transporting layer in accordance with a well known process such as the vacuum vapor deposition process, the spin coating process, the casting process and the LB process. Although the thickness of the hole injecting/transporting layer is not particularly limited, the thickness is usually from 5 nm to 5 μm. The hole injecting/transporting layer may be constructed by a layer comprising at least one of the aforementioned materials or by laminating a hole injecting/transporting layer comprising a different compound other than the aforementioned hole injecting/transporting layer.

In the organic EL device of the present invention, the organic semiconductor layer assists to inject the holes or to inject the electrons into the light emitting layer, and it is preferable for the organic semiconductor layer to have a electric conductivity of 10⁻¹⁰ S/cm or greater. With regard to a material for the organic semiconductor layer, electroconductive oligomers such as an oligomer having thiophene, an oligomer having arylamine disclosed in Japanese Unexamined Patent Application Laid-Open No. Hei 8-193191 and the like, electroconductive dendrimers such as a dendrimer having an arylamine dendrimer are employable.

VI. Electron Injecting Layer

The electron injecting layer in the organic EL device of the present invention is a layer which assists injection of electrons into the light emitting layer and exhibits a great mobility of electrons. In the electron injecting layer, an adhesion improving layer is a layer made of a material exhibiting excellent adhesion with the cathode. As the material for the electron injecting layer, metal complexes of 8-hydroxyquinoline and derivatives thereof are preferable.

Examples of metal complexes of 8-hydroxyquinoline and derivatives thereof include metal chelates of oxinoid compounds including chelates of oxine (in general, 8-quinolinol or 8-hydroxyquinoline).

For example, tris(8-quinolinolato)aluminum (Alq) can be employed as the electron injecting material.

Further, examples of the oxadiazole derivatives include an electron transfer compound shown as the following general formulae:

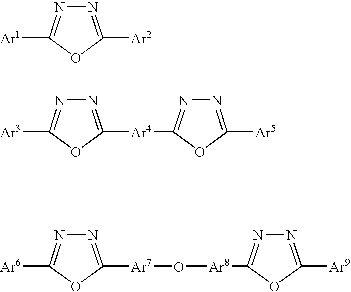

In the general formulae above, Ar¹, Ar², Ar³, Ar⁵, Ar⁶ and Ar⁹ each independently represents a substituted or unsubstituted aryl group respectively, which may be the same with or different from the other; Ar⁴, Ar⁷ and Ar⁸ each independently represents a substituted or unsubstituted arylene group, which may be the same with or different from the other. Examples of the aryl group include a phenyl group, a biphenyl group, an anthranil group, a perilenyl group and a pyrenyl group. Further, examples of the arylene group include a phenylene group, a naphthylene group, a biphenylene group, an anthranylene group, a perilenylene group, a pyrenylene group and the like. Furthermore, examples of the substituent include an alkyl group having 1 to 10 carbon atoms, an alkoxy group having 1 to 10 carbon atoms or a cyano group and the like. With regard to the electron transfer compounds, the compounds having a thin film forming capability are preferable.

Specific examples of the electron transfer compounds may be shown below:

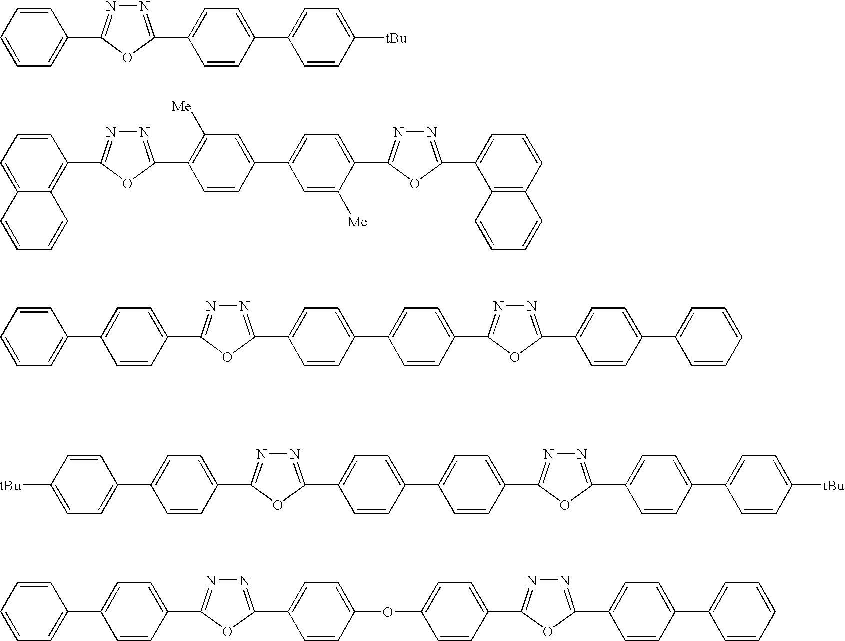

In addition, it has been known that compounds having a heterocyclic ring containing a nitrogen atom are suitable for an electron transporting material. Examples thereof include the following heterocyclic derivatives containing a nitrogen atom.

A preferable heterocyclic derivative containing a nitrogen atom includes the following structure:
HAr-L-Ar¹-Ar²

In the above general formula, HAr represents a heterocyclic group having carbon atoms of 3 to 40 and a nitrogen atom which may have substituent; L represents a single bond, a arylene group having carbon atoms of 6 to 60 which may have substituent, a heteroarylene group having carbon atoms of 6 to 60 which may have substituent, or a fluorenylene group which may have substituent; Ar¹ represents a divalent aromatic hydrocarbon group having carbon atoms of 6 to 60 which may have substituent; and Ar² represents a aryl group having carbon atoms of 6 to 60 which may have substituent, or a heteroaryl group having carbon atoms of 3 to 60 which may have substituent.

Further, the heterocyclic derivatives containing a nitrogen atom represented by any one of the following two general formulae are preferable.

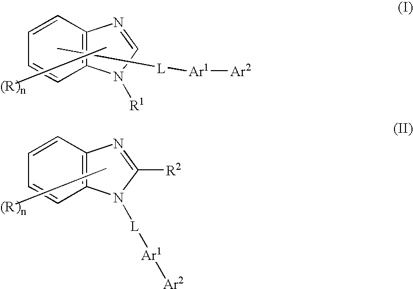

In the above general formulae, R represents a hydrogen atom, an aryl group having carbon atoms of 6 to 60 which may have substituent, a pyridyl group which may have substituent, a quinolyl group which may have substituent, an alkyl group having carbon atoms of 1 to 20 which may have substituent, or an alkoxy group having carbon atoms of 1 to 20 which may have substituent. n represents an integer of 0 to 4. R¹ represents an aryl group having carbon atoms of 6 to 60 which may have substituent, a pyridyl group which may have substituent, a quinolyl group which may have substituent, an alkyl group having carbon atoms of 1 to 20 which may have substituent, or an alkoxy group having carbon atoms of 1 to 20 which may have substituent. R² represents a hydrogen atom, an aryl group having carbon atoms of 6 to 60 which may have substituent, a pyridyl group which may have substituent, a quinolyl group which may have substituent, an alkyl group having carbon atoms of 1 to 20 which may have substituent, or an alkoxy group having carbon atoms of 1 to 20 which may have substituent. L represents an arylene group having carbon atoms of 6 to 60 which may have substituent, a pyridinylene group which may have substituent, a quinolinylene group which may have aubstituent, or a fluorenylene group which may have substituent. Ar¹ represents an arylene group having carbon atoms of 6 to 60 which may have substituent, a pyridinylene group which may have substituent, or a quinolinylene group which may have substituent. Ar² represents an aryl group having carbon atoms of 6 to 60 which may have substituent, a pyridyl group which may have substituent, a quinolyl group which may have substituent, an alkyl group having carbon atoms of 1 to 20 which may have substituent, or an alkoxy group having carbon atoms of 1 to 20 which may have substituent.

As a preferable embodiment of the present invention, there is a device that a reductive dopant is added in either the electron transporting zone or an interfacial zone between the cathode and the organic layer thereof. The reductive dopant used in the present invention is defined as a substance which reduces the electron transporting compound. Therefore, various compounds may be employed if they have a certain level of reduction capability, and examples of the preferable reductive dopant include at least one compound selected from the group comprising alkali metals, alkaline earth metals, rare earth metals, alkali metal oxides, alkali metal halides, alkaline earth metal oxides, alkaline earth metal halides, rare earth metal oxides, rare earth metal halides, organic complexes of alkali metals, organic complexes of alkaline earth metals and organic complexes of rare earth metals.

Examples of the more preferable reductive dopant include at least one alkali metal selected from a group consisting of Na (the work function: 2.36 eV), K (the work function: 2.28 eV), Rb (the work function: 2.16 eV) and Cs (the work function: 1.95 eV) or at least one alkaline earth metals selected from a group consisting of Ca (the work function: 2.9 eV), Sr (the work function: 2.0 to 2.5 eV) and Ba (the work function: 2.52 eV); whose work function of 2.9 eV or smaller is particularly preferable.

Among those, more preferable reductive dopants include at least one kind or more alkali metal selected from the group consisting of K, Rb and Cs, the latter Rb or Cs being farther more preferable and the last Cs being the most preferable. Those alkali metals have particularly high reducing capability, and only an addition of relatively small amount of them into an electron injecting zone enables to achieve both improvement of luminance and lifetime extension of the organic EL device. Further, with regard to the reductive dopant with work function of 2.9 eV or smaller, a combination of two or more kinds of the alkali metal is also preferable, and particularly, combinations containing Cs, for example, combinations of Cs and Na, Cs and K, Cs and Rb, or Cs and Na and K are preferable. Containing Cs in combination enables to reveal reducing capability effectively, and the addition into the electron injecting zone expects both improvement of luminance and lifetime extension of the organic EL device.

In the organic EL device of the present invention, an electron injecting layer formed with an insulating material or a semiconductor may be further sandwiched between the cathode and the organic thin film layer. The electron injecting layer effectively prevents leak in the electric current and improves the electron injecting capability. It is preferable that at least one metal compound selected from the group consisting of alkali metal chalcogenides, alkaline earth metal chalcogenides, alkali metal halides and alkaline earth metal halides is used as the insulating material. It is preferable that the electron injecting layer is constituted with the above alkali metal chalcogenide since the electron injecting capability can be improved. Preferable examples of the alkali metal chalcogenide include Li₂O, LiO, Na₂S, Na₂Se and NaO. Preferable examples of the alkaline earth metal chalcogenide include CaO, BaO, SrO, BeO, BaS and CaSe. Preferable examples of the alkali metal halide include LiF, NaF, KF, LiCl, KCl and NaCl. Preferable examples of the alkaline earth metal halide include fluorides such as CaF2, BaF2, SrF2, MgF2 and BeF2 and halides other than the fluorides.

Examples of the semiconductor constituting the electron transporting layer include oxides, nitrides and oxide nitrides containing at least one element selected from Ba, Ca, Sr, Yb, Al, Ga, In, Li, Na, Cd, Mg, Si, Ta, Sb and Zn, which are used singly or in combination of two or more. It is preferable that the inorganic compound constituting the electron transporting layer is in the form of a fine crystalline or amorphous insulating thin film. When the electron transporting layer is constituted with the above insulating thin film, a more uniform thin film can be formed and defective pixels such as dark spots can be decreased. Examples of the inorganic compound include the alkali metal chalcogenides, the alkaline earth metal chalcogenides, the alkali metal halides and the alkaline earth metal halides which are described above.

VII Cathode

As the cathode for the organic EL device of the present invention, an electrode substance such as metal, alloy, electroconductive compound and those mixture having a small work function (4 eV or smaller) is employed. Examples of the electrode substance include potassium, sodium- potassium alloy, magnesium, lithium, magnesium-silver alloy, aluminum/aluminum oxide, aluminum-lithium alloy, indium, rare earth metal, etc.

The cathode can be prepared by forming a thin film of the electrode material described above in accordance with a process such as the vapor deposition process and the sputtering process.

When the light emitted from the light emitting layer is obtained through the cathode, it is preferable that the cathode has a transmittance of the emitted light greater than 10%.

It is also preferable that the sheet resistivity of the cathode is several hundred Ω/□ or smaller. The thickness of the cathode is, in general, selected in the range of 10 nm to 1 μm and preferably in the range of 50 to 200 nm.

VIII. Insulating Layer

An organic EL device tends to form defects in pixels due to leak and short circuit since an electric field is applied to ultra-thin films. To prevent the formation of the defects, a layer of an insulating thin film is preferably inserted between the pair of electrodes.

Examples of the material employed for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide and vanadium oxide.

Mixtures and laminates of the above compounds may also be employed.

IX. Examples of Producing Organic EL Device

To produce an organic EL device of the present invention, for example, an anode, a light emitting layer and, where necessary, a hole injecting layer, and when necessary, an electron injecting layer may be formed in accordance with the aforementioned process using the aforementioned materials, and a cathode is formed in the last step. An organic EL device may be produced by forming the aforementioned layers in the order reverse to that described above, i.e., a cathode being formed in the first step and an anode in the last step.

An embodiment of the process for producing an organic EL device having a construction in which an anode, a hole injecting layer, a light emitting layer, an electron injecting layer and a cathode are disposed sequentially on a transparent substrate will be described in the following.

On a suitable transparent substrate, a thin film made of a material for the anode is formed in accordance with the vapor deposition process or the sputtering process so that the thickness of the formed thin film is 1 μm or smaller and preferably in the range of 10 to 200 nm. The formed thin film is employed as the anode. Then, a hole injecting layer is formed on the anode. The hole injecting layer can be formed in accordance with the vacuum vapor deposition process, the spin coating process, the casting process or the LB process, as described above. The vacuum vapor deposition process is preferable since a uniform film can be easily obtained and the possibility of formation of pin holes is small. When the hole injecting layer is formed in accordance with the vacuum vapor deposition process, in general, it is preferable that the conditions in general are suitably selected in the following ranges: temperature of the deposition source: 50 to 450° C.; vacuum level: 10-7 to 10-3 Torr; deposition rate: 0.01 to 50 nm/second; temperature of the substrate: −50 to 300° C.; and film thickness: 5 nm to 5 μm; although the conditions of the vacuum vapor deposition are different depending on the employed compound (the material for the hole injecting layer) and the crystal structure and the recombination structure of the hole injecting layer to be formed.

Subsequently, the light-emitting layer is formed on the hole-injecting layer formed above. Also the formation of the light emitting layer can be made by forming the desired light emitting material into a thin film in accordance with the vacuum vapor deposition process, the sputtering process, the spin coating process or the casting process. The vacuum vapor deposition process is preferable because a uniform film can be easily obtained and the possibility of formation of pinholes is small. When the light emitting layer is formed in accordance with the vacuum vapor deposition process, in general, the conditions of the vacuum vapor deposition process can be selected in the same ranges as those described for the vacuum vapor deposition of the hole injecting layer although the conditions are different depending on the used compound.

Next, the electron injecting layer is formed on the light emitting layer formed above. Similarly to the hole injecting layer and the light emitting layer, it is preferable that the electron injecting layer is formed in accordance with the vacuum vapor deposition process since a uniform film should be obtained. The conditions of the vacuum vapor deposition can be selected in the same ranges as those for the hole injecting layer and the light emitting layer.

Although the aromatic amine derivatives of the present invention depend on that it is contained in a light emitting zone or a hole transporting zone, it may be vapor deposited together with other materials. In addition, when the spin coating process is employed, it may be contained therein by blending it with other materials.

An organic EL device is produced by laminating a cathode as the last step. The anode is made of a metal and can be formed in accordance with the vacuum vapor deposition process or the sputtering process. It is preferable that the vacuum vapor deposition process is employed in order to prevent the lower organic layers from damages during the formation of the film.

In the above production of the organic EL device, it is preferable that the above layers from the anode to the cathode are formed successively while the production system is kept in a vacuum after being evacuated once.

The process for forming the layers in the organic EL device of the present invention is not particularly limited. A conventional process such as the vacuum vapor deposition process and the spin coating process can be used. The organic thin film layer comprising the compound represented by the foregoing general formula (1) used in the organic EL device of the present invention can be formed in accordance with the vacuum vapor deposition process, the molecular beam epitaxy process (the MBE process) or, using a solution prepared by dissolving the compound into a solvent, in accordance with a conventional coating process such as the dipping process, the spin coating process, the casting process, the bar coating process and the roller coating process.

The thickness of each layer in the organic thin film layer in the organic EL device of the present invention is not particularly limited. In general, an excessively thin layer tends to have defects such as pin holes, and an excessively thick layer requires a high applied voltage results in decreasing the efficiency. Therefore, a thickness within the range of several nanometers to 1 μm is preferable.

The organic EL device which can be produced as described above emits light when a direct voltage of 5 to 40 V is applied in the condition that the anode is connected to a positive electrode (+) and the cathode is connected to a negative electrode (−). When the connection is reversed, no electric current is observed and no light is emitted at all. When an alternating voltage is applied to the organic EL device, the uniform light emission is observed only in the condition that the polarity of the anode is positive and the polarity of the cathode is negative. When an alternating voltage is applied to the organic EL device, any type of wave shape can be employed.

### EXAMPLE

This invention will be described in further detail with reference to the examples, which do not limit the scope of this invention.

### Example 1 (Synthesis of TA-1)

(1) Synthesis of N-biphenyl-N-phenylamine

Under the argon gas current, 126 g of 4-bromobiphenyl (manufactured by Lancaster Synthesis Co., Ltd.), 65 g of acetanilide (manufactured by Wako Pure Chemical Industries, Ltd.), 75 g of potassium carbonate (manufactured by Wako Pure Chemical Industries, Ltd.), 3.5 g of copper powder (manufactured by Wako Pure Chemical Industries, Ltd.) and 500 ml of decalin (manufactured by Wako Pure Chemical Industries, Ltd.) were placed, and then they were reacted at 200° C. for 6 days.

After the reaction was completed, it was cooled down, and toluene was added therein, followed by filtration to obtain insoluble. The insoluble matter was dissolved in chloroform, followed by removing insoluble, and then the resultant was treated with activated carbon, followed by concentration of the solution. Acetone was added in the resultant, and the precipitated crystal was obtained by filtration.

The crystal obtained was suspended in 500 ml of ethylene glycol (manufactured by Wako Pure Chemical Industries, Ltd.) and 500 ml of water, and then 36 g of 85% potassium hydroxide aqueous solution added therein, followed by carrying out the reaction at 120° C. for 2 hours.

After the reaction was completed, the resultant was poured in 1 liter of water and the precipitated crystal was obtained by filtration. Subsequently, it was washed by water and methanol.

The crystal obtained was dissolved in tetrahydrofuran while heating, followed by treating the solution with activated carbon. Subsequently, the crystal was precipitated by adding acetone therein. The precipitated crystal was filtrated and 75 g of N-biphenyl-N-phenylamine was obtained.

(2) Synthesis of N-biphenyl-N-phenyl-4-amino-4′-iodo-1,1′-biphenyl

50 g of the obtained N-biphenyl-N-phenylamine, 83 g of 4,4′-diiodobiohenyl (manufactured by Tokyo Kasei Kogyo Co. Ltd.), 30 g of potassium carbonate (manufactured by Wako Pure Chemical Industries, Ltd.), 1.5 g of copper powder (manufactured by Wako Pure Chemical Industries, Ltd.) and 500 ml of decalin (manufactured by Wako Pure Chemical Industries, Ltd.) were placed, and then the reaction was carried out at 200° C. for 6 days.

After the reaction was completed, it was filtrated during hot, and then the insoluble was washed by toluene. The both filtrated solutions were concentrated together. Toluene was added in the residue and the precipitated crystal was removed by filtration, followed by concentrating the filtrate. Subsequently, methanol was added in the residue, followed by stirring and disposing the supernatant liquid. Further, methanol was added therein, followed by stirring and disposing the supernatant liquid. The yellow powder was obtained through column refining of the residue. It was dissolved in toluene while heating, and hexane was added therein, followed by cooling down to precipitate crystal. The crystal obtained by filtration was 40 g of N-biphenyl-N-phenyl-4-amino-4′-iodo-1,1′-biphenyl.

(3) Synthesis of TA-1

Under the argon gas current, 40 g of N-biphenyl-N-phenyl-4-amino-4′-iodo-1,1′-biphenyl, 10 g of N,N′-diphenyl-4,4′-benzidine, 10 g of potassium carbonate (manufactured by Wako Pure Chemical Industries, Ltd.), 0.4 g of copper powder (manufactured by Wako Pure Chemical Industries, Ltd.) and 1 liter of decalin (manufactured by Wako Pure Chemical Industries, Ltd.) were placed, and then the reaction was carried out at 200° C. for 6 days.

After the reaction was completed, it was filtrated during hot, and then the insoluble was washed by toluene. The both filtrated solutions were concentrated together. Toluene was added in the residue and the precipitated crystal was removed by filtration, followed by concentrating the filtrate. Subsequently, methanol was added in the residue, followed by stirring and then disposing the supernatant liquid. Further, methanol was added therein, followed by stirring and then disposing the supernatant liquid. The yellow powder was obtained through column refining of the residue. It was dissolved in toluene on heating, and hexane was added therein, followed by cooling down to precipitate crystal which was obtained by filtration.

By sublimating the crystal obtained, 7.7 g of pale yellow powder was obtained.

The measurement result of the compound by FD-MS (Field Desorption Mass Spectrometry analysis) showed the main peak of m/z (measured value)=1127 to C₈₄H62N4=1126, therefore TA-1 was confirmed.

### Example 2 (Synthesis of TA-6)

(1) Synthesis of 4-bromo-4′-iodobiphenyl

50.0 g of 4-bromobiphenyl, 23.7 g of iodine, 10.6 g of ortho periodicacid, 13 ml of concentrated sulfuric acid, 400 ml of acetic acid and 45 ml of water were placed, and then they were stirred at 90° C. for 7 hours. After the reaction was completed, it was cooled down to room temperature, and then 1 liter of water was poured therein, followed by stirring for 1 hour. The precipitated solid was separated by filtration, followed by methanol washing and drying under reduced pressure, and then 68.0 g of 4-bromo-4′-iodobiphenyl as white crystal was obtained.

(2) Synthesis of 4-(N,N-diphenylamino)-4′-bromobiphenyl

15.7 g of 4-bromo-4′-iodobiphenyl, 7.44 g of N,N-diphenylamine, 1.67 g of copper(I) iodide, 6.31 g of sodium t-butoxide, 772 mg of N,N′-dimethylethylene-diamine and 50 ml of xylene were placed, and then they were stirred for 18 hours under reflux. The resultant was cooled down to room temperature, followed by extraction by using 500 ml of toluene and 300 ml of water, and then insoluble was. removed by filtration. After the water layer was removed, the organic layer was dried with the use of magnesium sulfate, and then the solvent was removed by distillation under reduced pressure. The residue was refined through a silica gel chromatography and then 13.5 g of 4-(N,N-diphenylamino)-4′-bromobiphenyl as white crystal was obtained.

(3) Synthesis of N-(diphenyl-4-yl)-N′-phenyl-4,4′-benzidine

Under the argon gas current, 208 g of N-acetyl-4-aminobiphenyl, 400 g of 4, 4′-diiodobiphenyl (manufactured by Wako Pure Chemical Industries, Ltd.), 204 g of potassium carbonate (manufactured by Wako Pure Chemical Industries, Ltd.), 12.5 g of copper powder (manufactured by Wako Pure Chemical Industries, Ltd.) and 2 liters of decalin were placed, and then they were reacted at 190° C. for 3 days.

After the reaction was completed, it was cooled down and 2 liters of toluene was added therein, followed by filtration to obtain insoluble. The unfiltered was solved in 4.5 liter of chloroform, followed by removing the insoluble, and then the resultant was treated with activated carbon, followed by concentration of the filtrate. The resultant was added with 3 liters of acetone, ant then 307 g of 4-(N-acetyl-(N-diphenyl-4-yl) amino)-4′-iodobiphenyl was obtained by filtration.

Subsequently, under the argon gas current, 290 g of 4-(N-acetyl-(N-diphenyl-4-yl)amino)-4′-iodobiphenyl, 160 g of acetanilide (manufactured by Wako Pure Chemical Industries, Ltd.), 165 g of potassium carbonate (manufactured by Wako Pure Chemical Industries, Ltd.), 12.5 g of copper powder (manufactured by Wako Pure Chemical Industries, Ltd.) and 2 liters of decalin were placed, and then they were reacted at 190° C. for 4 days.

After the reaction was completed, it was cooled down and 2 liters of toluene was added therein, followed by filtration to obtain insoluble. The unfiltered was dissolved in 4.5 liter of chloroform, followed by removing the insoluble, and then the resultant was treated with activated carbon, followed by concentration of the filtrate. 3 liters of acetone was added therein and the precipitated crystal was removed by filtration.

The resultant was suspended in 5 liters of ethylene glycol (manufactured by Wako Pure Chemical Industries, Ltd.), and 50 ml of water, and then 145 g of 85% potassium hydroxide aqueous solution added therein, followed by carrying out the reaction at 120° C. for 2 hours.

After the reaction was completed, the resultant was poured in 10 liters of water, followed by separating the precipitated crystal through filtration, and then it was washed with water and methanol.

The resultant crystal was dissolved in 3 liters of tetrahydrofuran on heating, and then the resultant was treated with activated carbon, followed by concentration of the filtrate. Subsequently, the crystal was precipitated by adding acetone therein. The crystal was obtained by filtration and 164 g of N-(diphenyl-4-yl)-N′-phenyl-4,4′-benzidine was obtained.

(4) Synthesis of TA-6 (N,N′-bis [4′-(N,N-diphenylamino)biphenyl-4-yl]-N-(diphenyl-4-yl)-N′-phenylbenzidine

In 100 ml of toluene solution containing 8.4 g of 4-(N,N-diphenylamino)-4′-bromobiphenyl, 3.94 g of N-(diphenyl-4-yl)-N′-phenyl-4,4′-benzidine, 437 mg of tris(dibenzylideneacetone)dipalladium and 2.14 g of sodium t-butoxide, 154 μl of toluene solution containing 50 wt % of t-butylphosphine was added, and then it was stirred at 80° C. for 18 hours. After the reaction was completed, the mixture was filtrated through cerite, and then the filtrate was concentrated. The residue was refined through a silica gel chromatography, followed by methanol washing of the crystal obtained, and then 9.51 g of the objective compound as pale yellow powder (TA-6) was obtained.

The measurement result of the compound by FD-MS showed m/z=1051 to 1050 of molecular weight, therefore TA-6 was confirmed.

### Example 3 (Evaluation of TA-1)

A glass substrate (manufactured by GEOMATEC Company) of 25 mm×75 mm×1.1 mm thickness having an ITO transparent electrode was cleaned by application of ultrasonic wave in isopropyl alcohol for 5 minutes and then by exposure to ozone generated by ultraviolet light for 30 minutes.

The cleaned glass substrate having an ITO transparent electrode line was fixed to a substrate holder of a vacuum deposition apparatus, and on the surface, where the ITO transparent electrode line was fixed, of the substrate, a film layer having film thickness of 80 nm of TA-1 was formed so as to cover the transparent electrode. The film performs as a hole transporting layer.

Subsequently, a layer having layer thickness of 40 nm of EM1 was formed through a vapor deposition. Concurrently, as a light emitting molecule, the following amino compound D1 containing a styryl group was deposited at the ratio by weight between EM1 and D1 of 40:2 by a vapor deposition. The film performs as a light emitting layer.

On the film, a film having an Alq film thickness of 10 nm was formed. The film performs as an electron injecting layer. Further, a film (film thickness: 10 nm) of Alq: Li (the source of lithium: manufactured by SAES GETTERS Company) as an electron injecting layer or a cathode was formed by binary vapor deposition of Li as a reductive dopant and the following Alq. On the Alq: Li film, Al metal was deposited to form a metal cathode, therefore, an organic EL device was fabricated.

The half lifetime of the organic EL device was measured at the initial luminance of 5,000 nit, room temperature and DC constant current driving. The results are shown in Table 1.

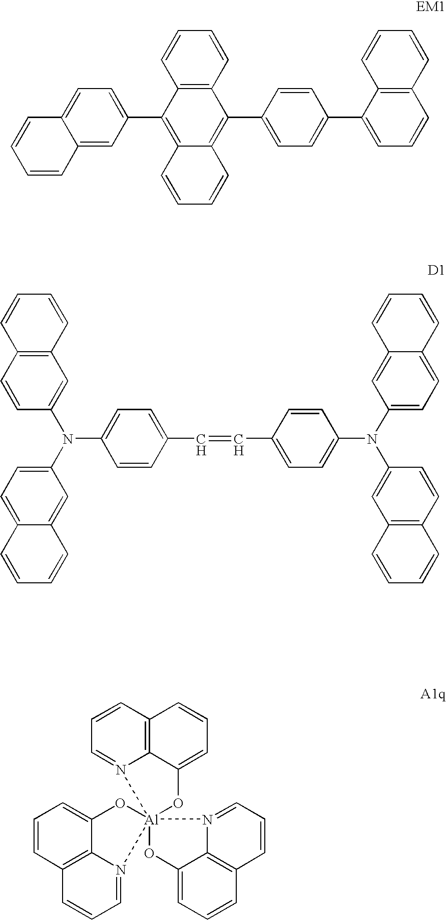

### Example 4 (Evaluation of TA-6)

The same procedure of Example 3 was repeated except that TA-6 in place of TA-1 was used, and the organic EL device was fabricated.

The half lifetime of the organic EL device was measured at the initial luminance of 5,000 nit, room temperature and DC constant current driving. The results are shown in Table 1.

### Comparative Example 1 (Evaluation of ta-1)

The same procedure of Example 3 was repeated except that ta-1 in place of TA-1 was used, and the organic EL device was fabricated.

The half lifetime of the organic EL device was measured at the initial luminance of 5,000 nit, room temperature and DC constant current driving. The results are shown in Table 1.

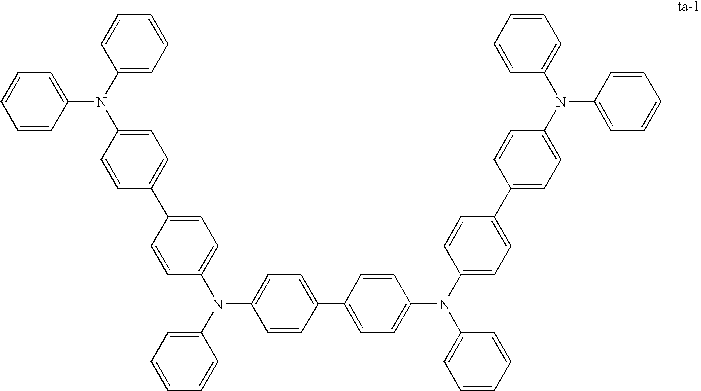

### Comparative Example 2 (Evaluation of ta-2)

The same procedure of Example 3 was repeated except that ta-2 in place of TA-1 was used, and the organic EL device was fabricated.

The half lifetime of the organic EL device was measured at the initial luminance of 5,000 nit, room temperature and DC constant current driving. The results are shown in Table 1.

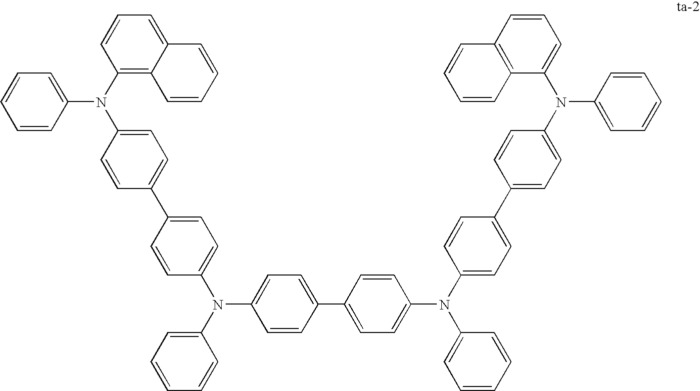

### Comparative Example 3 (Evaluation of ta-3)

The same procedure of Example 3 was repeated except that ta-3 in place of TA-1 was used, and the organic EL device was fabricated.

The half lifetime of the organic EL device was measured at the initial luminance of 5,000 nit, room temperature and DC constant current driving. The results are shown in Table 1.

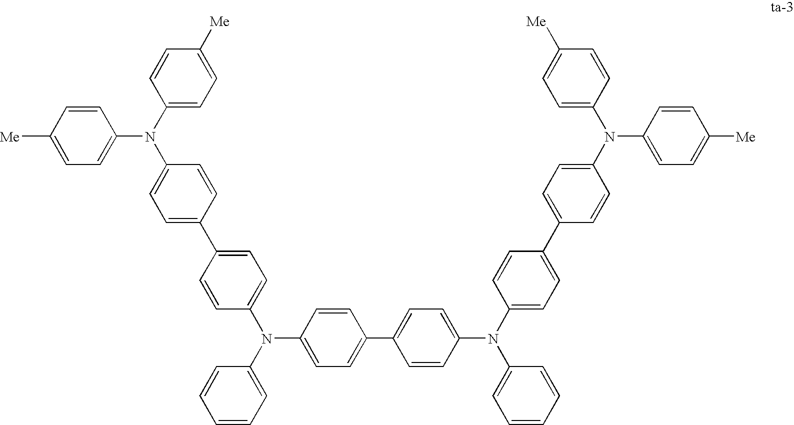

TABLE 1

| | | Half Lifetime (h) | |
|---|---|---|---|
| | Hole | at Initial | |
| | Transporting | Luminance of | Emitted |
| | Material | 5,000 nit | Color |
| Example 3 | TA-1 | 430 | Blue |
| Example 4 | TA-6 | 450 | Blue |
| Comparative Example 1 | ta-1 | 120 | Blue |
| Comparative Example 2 | ta-2 | 180 | Blue |
| Comparative Example 3 | ta-3 | 80 | Blue |

As shown from the above results, when the aromatic amine derivatives of the present invention are used for a hole transporting material of an organic EL device, it is found that the decay of luminance of the organic EL device based on driving it is low, and in particular, improvement of decaying luminance based on driving a device is notable at a device emitting blue light.

### INDUSTRIAL APPLICABILITY

As explained in details, an organic EL device including the aromatic amine derivatives of the present invention exhibits various luminescent hue and has high heat resistance. An organic EL device including the aromatic amine derivatives as a hole injecting/transporting material exhibits high a hole injecting/transporting capability, high luminance and high current efficiency, and also has a long lifetime due to low decay of luminance based on driving the device.

Therefore, the organic EL device of the present invention is significantly suitable for a practical use and can be useful for a flat light emitter for a television hanging on walls, a light source for a backlight of displays and the like. The aromatic amine derivatives may be used as a charge transporting material for electrophotography conductor and a organic semi-conductor as well as an organic EL device and a hole injecting/transporting material. The advantages of the organic EL device can be particularly demonstrated on a device emitting blue light.

What we claim is:

1. An aromatic amine derivative represented by the general formula (I): 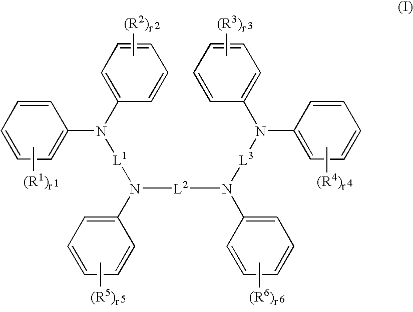
wherein R¹ to R⁶ each independently represents a substituted or unsubstituted alkyl group having carbon atoms of 1 to 6, or a substituted or unsubstituted aryl group having nuclear carbon atoms of 6 to 20, L¹ to L³ each independently represents an linking group represented by the general formula (II); 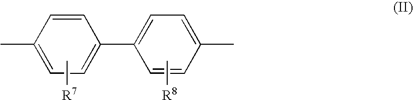
wherein R⁷ and R⁸ each independently represents a hydrogen atom, a substituted or unsubstituted alkyl group having carbon atoms of 1 to 6, or a substituted or unsubstituted aryl group having nuclear carbon atoms of 6 to 20, in addition, R⁷ and R⁸ may bond each other to form a saturated or unsaturated ring; r¹ to r⁶ each independently represents an integer of 0 to 5, and r¹+r²+r³+r⁴+r⁵+1, further, when any one of r¹ to r⁶ is 2 or larger, each of R¹ to R⁶ corresponding thereto may be the same with or different from the other;
however, at least one of R¹ to R⁶ is a substituted or unsubstituted aryl group having nuclear carbon atoms of 6 to 20.

2. The aromatic amine derivative according to claim 1, wherein L¹ to L³ of linking groups each independently is selected from the following general formulae (II-1) to (II-4): 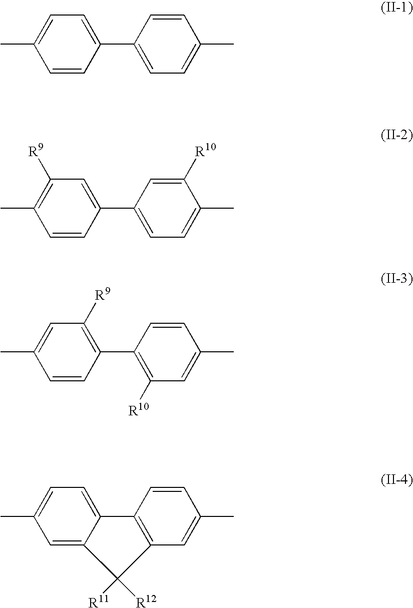
wherein R⁹ to R¹² each independently represents a substituted or unsubstituted alkyl group having carbon atoms of 1 to 6, or a substituted or unsubstituted aryl group having nuclear carbon atoms of 6 to 20;
however, R¹¹ and R¹² may bond each other to form a saturated or unsaturated ring.

3. The aromatic amine derivative according to claim 1, wherein the aromatic amine derivative is a material for an organic electroluminescence device.

4. An organic electroluminescence device which comprises at least one organic thin film layer comprising a light emitting layer sandwiched between a pair of electrode consisting of an anode and a cathode, wherein at least one of the organic thin film layers comprises the aromatic amine derivative according to claims 1 or 2 singly or as a mixture component thereof.

5. The organic electroluminescence device according to claim 4, wherein the organic thin film layer comprises a hole transporting zone including the aromatic amine derivative.

6. The organic electroluminescence device according to claim 4, wherein the organic thin film layer comprises a hole transporting layer including the aromatic amine derivative.

7. The organic electroluminescence device according to claim 6, wherein the hole transporting layer comprises primarily the aromatic amine derivative.

8. The organic electroluminescence device according to claim 4, wherein the organic thin film layer comprises a layer of a hole transporting layer comprising the aromatic amine derivative and a light emitting layer comprising a phosphorescence metal complex and a host material.

9. The organic electroluminescence device according to any one of claims 4 to 8, wherein the device emits blue light.
